(19) United States
(12) Patent Application Publication
(10) Pub. No.: US 2025/0000434 A1
(43) Pub. Date: Jan. 2, 2025

(54) SYSTEM AND METHOD FOR MONITORING BRAIN TRAUMA EXPOSURE

(71) Applicant: BRAINWARE AI, INC., Rockliffe Park (CA)

(72) Inventors: Clara Karton, Ottawa (CA); Thomas Blaine Hoshizaki, Ottawa (CA); Ryan Graham, Kanata (CA); Gwyneth Ross, Newton, MA (US); Allison Clouthier, Ottawa (CA)

(73) Assignee: BRAINWARE AI, INC., Rockliffe Park, ON (CA)

(21) Appl. No.: 18/570,746

(22) PCT Filed: Sep. 8, 2022
(86) PCT No.: PCT/CA2022/051345

§ 371 Date: Dec. 15, 2023

Related U.S. Application Data
(60) Provisional application No. 63/241,600, filed on Sep. 8, 2021.

(51) Int. Cl. A61B 5/00; A61B 5/11

(52) U.S. Cl. CPC A61B 5/4064; A61B 5/1128; A61B 5/7267; A61B 5/7275; A61B 2503/10

(57) ABSTRACT

The present disclosures provides for a system and a method for evaluating brain trauma exposure from a motion capture source. Preferably, the system is comprised of a motion capture source engaged with a non-transitory computer readable medium such as a processor. The motion capture source is adapted to view or record images or videos of human movement. The processor determines and extracts key characteristics of a head impact and predicts a brain response based on such characteristics. The system is then able to generate and provide a brain trauma assessment output based on the brain response. A method is also disclosed, which extracts key characteristics from the motion capture source, predicts a brain response based on such key characteristics and provides a brain trauma exposure output based on the brain response.

### FIELD

The present disclosure generally relates to quantifying single and cumulative brain trauma, and, more specifically, the prediction of brain response, using artificial intelligence and data analytics to monitor and manage exposure and brain health.

### BACKGROUND

Exposure to brain trauma from sport, physical activity, military and the workplace is a public health concern. A predominant challenge in injury prediction and risk mitigation is that approaches that rely on concussion symptoms as a marker for injury have been largely ineffective. Physical trauma to the brain affects the integrity of the neurological system leading to acute and chronic impairments, not limited to concussion symptoms, but rather manifest in changes in cognition, behaviour and psychology. Further, this is partly the result of attempting to connect inconsistent and subjective symptoms and individualized expression of brain trauma to a number of subtle, less obvious independent brain responses. Brain trauma causes a number of physiological responses leading to injury ranging from acutely undetectable to neurodegenerative and catastrophic brain damage. The brain's responses to traumatic forces are difficult to measure in vivo and therefore have been inherently difficult to quantify, especially in real-time. Consequently, the spectrum of brain trauma, including acutely asymptomatic impacts and changes to mental health associated with detectable changes to neurons remain unmonitored in real-time. To accurately protect, prevent, and treat head and brain injury, an effective and objective brain trauma exposure measurement tool is required. Brain motion upon impact is influenced by a combination of head impact event conditions such as the speed, the head impact location and direction, and the masses and materials involved in the impact. The interaction of these parameters characterize how and to what degree the head and brain move from the impact, subsequently causing damage and injury. Manually obtaining event parameters from video to inform physical reconstructions and the use of sophisticated brain finite element analysis provides a gold standard for accurately measuring brain tissue motion. This process however is labour intensive requiring technical expertise and equipment making it inaccessible to the general population and other non-expert user groups.

There is a great need for an effective and accurate method for capturing the brain's response to trauma that can be accessible to anyone involved in sports, activities, or occupations where head trauma may occur. Predictive algorithms involving motion capture combined with statistical and artificial intelligence methods can be used to automate this process to obtain brain kinematic and kinetic measures to create an economical and effective tool to capture and quantify brain trauma exposure in real-time. The creation of large datasets of brain trauma exposures for different environments, populations/demographics, and medical predisposition and history provides an opportunity for an individualized approach to risk management. The effects of brain trauma present unique risk to an individual and therefore requires individualized decision making and health care needs.

### SUMMARY

In an aspect, the present disclosure provides a method for evaluating brain trauma exposure, the steps comprising: extracting key characteristics from at least one motion capture source; predicting a brain response based on the key characteristics; and, providing a brain trauma assessment output based on the brain response, wherein at least one of statistical methods and machine learning models are utilized to predict the brain response and provide the brain trauma assessment.

In another aspect, the present disclosure provides a system for evaluating brain trauma exposure, the system comprising: at least one motion capture source; and, a non-transitory computer readable medium connected to the at least one motion capture source, the computer readable medium configured to: extract key characteristics from the at least one motion capture source; predict a brain response based on the key characteristics; and, provide a brain trauma assessment output based on the brain response wherein at least one of statistical methods and machine learning models are utilized to predict the brain response and provide the brain trauma assessment, and wherein the brain trauma exposure is provided to a user.

### BRIEF DESCRIPTION OF THE DRAWINGS

The following figures serve to illustrate various embodiments of features of the disclosure. These figures are illustrative and are not intended to be limiting.

FIG. 1 is a flowchart of a process for evaluating brain trauma exposure from a motion capture source in accordance with an embodiment of the present disclosure;

FIG. 2 is a flowchart of a process for predicting a brain response using a motion capture source in accordance with an embodiment of the present disclosure;

FIG. 3 is a diagram of a system for determining and evaluating brain trauma exposure using the motion capture source in accordance with an embodiment of the present disclosure;

FIG. 4 is an image of an athlete taken from an image capture source, the image capture source having identified key body and head elements on the athlete, in accordance with an embodiment of the present disclosure;

FIG. 5 is an image taken from an image capture source of an athlete actively on an ice rink, the image capture source using a Canny edge detector method to identify an outline of the athlete, in accordance with an embodiment of the present disclosure;

FIG. 6 is an image of an outline of an athlete on an ice rink showing a weighted centroid, the weighted centroid representing all the pixels within the outline of the athlete's helmet or head, using a process for detecting a physical head impact, in accordance with an embodiment of the present disclosure;

FIG. 7 is a graph showing coordinates of a set of elements measured over time from a plurality of images of a video, in accordance with an embodiment of the present disclosure;

FIG. 8 is a flowchart architecture of a decision tree model for predicting brain response using a set of characteristics extracted from the motion capture source in accordance with an embodiment of the present disclosure; and,

FIG. 9 is a funnel flowchart depicting a set of features that may be used to create individualized brain trauma assessments.

### DETAILED DESCRIPTION

The following embodiments are merely illustrative and are not intended to be limiting. It will be appreciated that various modifications and/or alterations to the embodiments described herein may be made without departing from the disclosure and any modifications and/or alterations are within the scope of the contemplated disclosure.

A preferred version of the present disclosure is an automated system for using artificial intelligence and data analytics to detect, measure and quantify physical head impact events causing trauma to the brain by sports participants, such as in ice hockey, football (soccer), American-style football, and rugby players among others. The automated system may further include a brain trauma exposure system that trains a model to evaluate head impacts and their characteristics, such as inbound velocity, location, and impact surfaces, through video (or images) captured by at least one image motion capture device. The automated system may further generate brain trauma assessments, such assessment configured to extract objective variables from video of head impacts sustained during sports and physical activity participation.

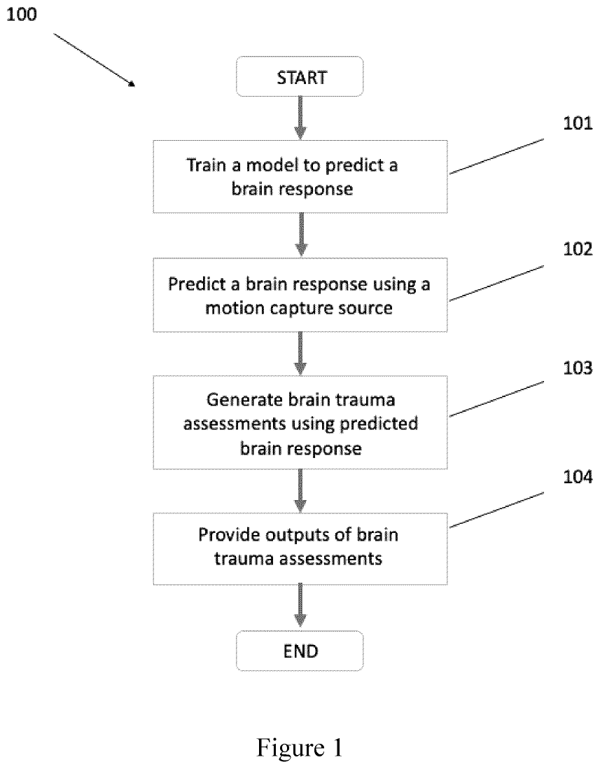

With reference to FIG. 1 and according to an embodiment of the present disclosure, a method 100 for evaluating brain trauma exposure is shown by means of a flowchart. In a first step 101, the method 100 trains a model to predict measurements of a brain response using key characteristics of head impact events. In a second step 102, the method 100 predicts a brain response using a motion capture source of individuals performing a set of movements, such as in a sport or physical activity. In a third step 103, the method 100 uses the predicted brain response to generate brain trauma assessments. In a fourth step 104, the method 100 outputs the generated brain trauma assessments as indicators of the brain trauma exposure. Each one of the first, second, third and fourth steps 101, 102, 103 and 104 of the method 100 shall be described in further detail below.

With further reference to FIG. 1, the first step 101 of training the model to predict a brain response is further described. A worker skilled in the art would appreciate that a brain response is any change in the brain associated with a physical, biological and/or metabolic measure or metric of, or associated with, neural tissue damage and/or head and brain injury, often resulting from motion or displacement of brain tissue. For example, the predicted brain response can be a metric of head or brain kinematic or kinetic measurements, such as displacement, deformation, acceleration, velocity, axonal strain, maximum principal strain, shear strain, von mises stress. The brain response may also be the integration of two or more head or brain kinematic or kinetic measurements, for example, Gadd Severity Index (GSI), Head Injury Criterion (HIC), Brain Injury Criterion (BrIC), Diffuse Axonal Multi-Axis General Evaluation (DAMAGE), Cumulative Strain Damage Measure (CSDM). In this first step, existing head impact and injury data are used to train one or multiple artificial intelligence models. Once trained, the model(s) is used to predict a brain response, caused from a variety of head motions. In this disclosure, head motion is defined as any motion of the head resulting from direct or indirect head and/or body impact. In other embodiments, both existing and real-time data are fed into the model(s) to improve the ability of the model(s) to predict the brain response. A worker skilled in the art will appreciate that the model(s) will be trained for the prediction of a particular brain response.

With further reference to step 101 of FIG. 1, a set of key characteristics are used as input to train a model to predict a brain response. In the present invention, the preferred set of key characteristics that are used to train the model are the parameters that describe a head impact event. Examples of head impact event parameters include, velocity of head impact, location on the head, direction of the impact, orientation of impact, type of impact event. The type of impact event is defined by the mass (e.g. head, body segments, projectiles) and material compliance of the impacting bodies/objects/surfaces (e.g. shoulder pad, turf, soccer ball) involved in the impact. In an alternate variation of the preferred embodiment, the set of key characteristics used to train a predictive model are head kinematic and/or kinetic variables. For instance, kinematic variables may relate to the components and/or waveforms describing head motion such as velocity, acceleration, time to peak, curve shape, and/or area under the curve.

With further reference to FIG. 1, the second step 102 of predicting a brain response using a motion capture source of individuals performing a set of movements is further described. The set of movements contemplated by the present disclosure can include, but is not limited to, an activity, sport, military service or training activity, and occupational or workplace activity. Motion capture sources contemplated by the present disclosure may include, but are not limited to, video camera, smart phone, electronic device, accelerometer, gyroscope, global positioning system, and inertial measurement unit. In a preferred embodiment, the key characteristics used to predict the brain response are extracted from a plurality of images captured from a video capture source. In another embodiment, the key characteristics are extracted using an impact detection tool acting as a motion capture source. In this alternate embodiment, the tool is an electronic sensor, mechanical device, orientation and/or navigation system. The type of tool would preferably be attached to a body or head segment, skin, and/or another wearable implement such as head gear, clothing etc.

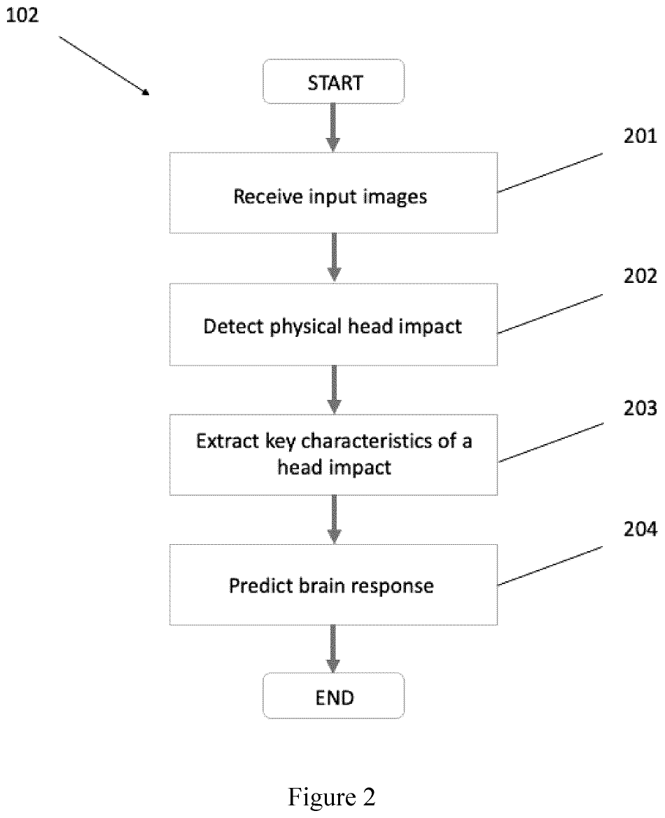

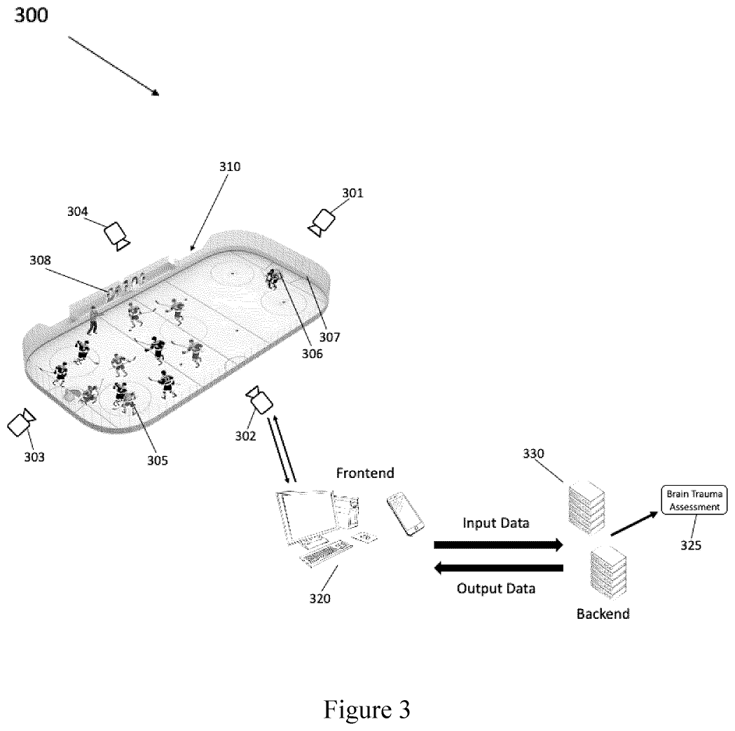

With reference to FIGS. 2 and 3 and according to an embodiment of the present disclosure, the second step 102 of predicting a brain response using a motion capture source is broken down into further sub-steps and described in further detail. A worker skilled in the art would appreciate that this second step 102 is performed by a processing device that can be either local or remote. Examples of such processing devices could include a local computer or mobile device but could also include remote processing on the cloud. For illustrative purposes, a computer 320 is used and shown. In a first sub-step 201, the computer 320 receives input images from the motion capture source. In a second sub-step 202, the computer 330 preprocesses the input images to detect a physical head impact event. In a third sub-step 203, the computer 330 extracts a set of key characteristics of the physical head impact event. In a fourth step sub-204, the computer 330 predicts the brain response using the extracted set of key characteristics. Each one of the first, second, third and fourth sub-steps 201, 202, 203 and 204 shall be described in further detail below.

With further reference to the first sub-step 201 of FIGS. 2 and 3, the computer 320 may receive a variety of inputs, including live or pre-recorded video of a user playing a sport, doing an activity, or otherwise performing a set of movements. The computer 320 may process images from a single video captured using a single perspective or may process images from multiple videos captured from different perspectives. The input may also be comprised of videos that have been previously recorded, such as in a game, match or practice of individuals participating in a sport or activity.

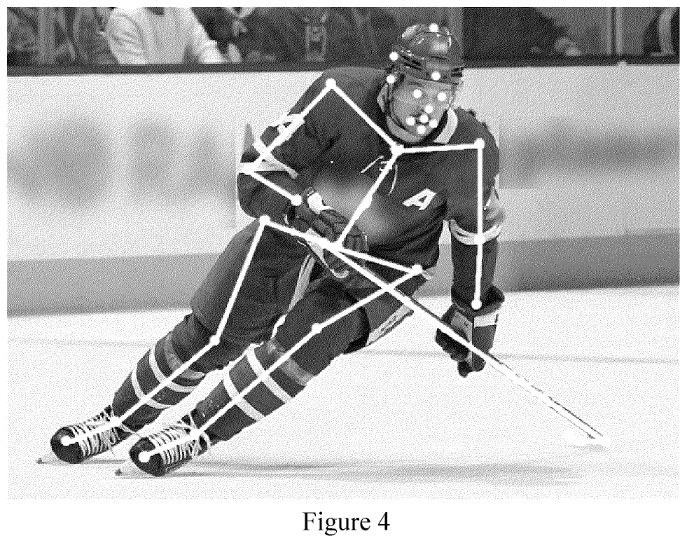

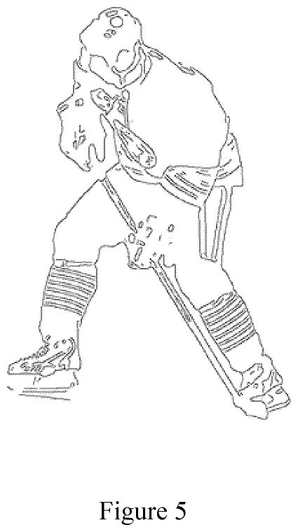

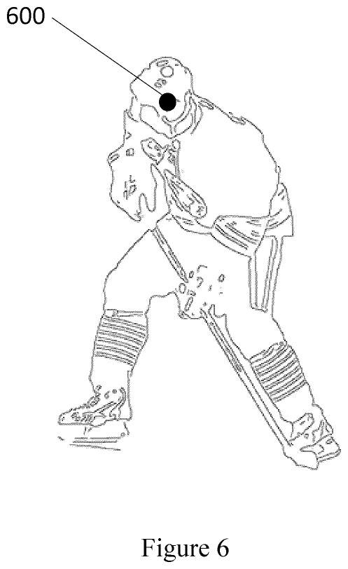

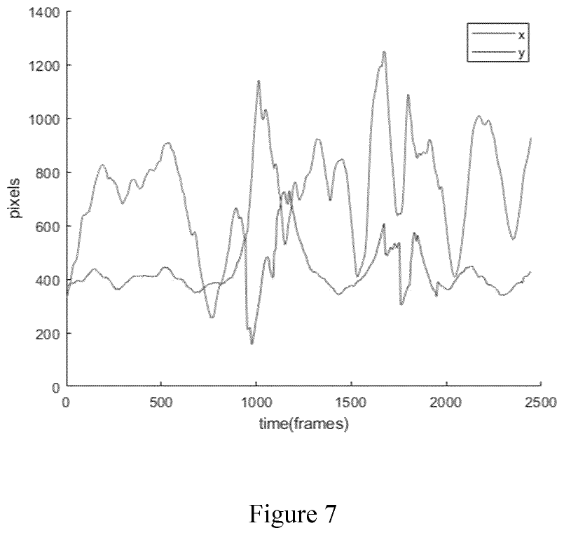

With further reference to the second sub-step 202 in FIG. 2, the position and trajectories, using 2D or 3D coordinates (i.e. set of elements), of head and body and objects and surfaces within an environment within one or multiple images, are used to detect a physical head impact. The set of elements can be identified using one or more statistical methods and/or machine learning models, such as convolutional neural networks (CNN), to automate the identification of individuals, objects and surfaces within an environment. Such identification may include a sports field or an arena, individual players, turf, ice, boards, posts, balls etc. This identification is accomplished by annotating a set of digital images by using a type of annotation, including but not limited to bounding boxes, polygonal segmentation, semantic segmentation, 3D cuboids, key-point and landmark, or lines and splines to label people, objects and surfaces of an environment within the set of digital images. The models are trained by providing a set of digital images with labeled individuals, objects and surfaces to one or more machine learning and/or statistical models. A worker skilled in the art would appreciate that examples of such set of elements are illustrated in FIG. 4. In this exemplary embodiment, head and body elements are identified in an image, each having x, y coordinates within the image. In a preferred embodiment, head impact detection is accomplished by computing the difference in position of a set of elements within a plurality of consecutive images that are captured in a video. This can be accomplished using a combination of time and frequency domain metrics (e.g. Teager-Kaiser energy operators), using real-world unit head/helmet coordinates, with various cut-off frequencies (determined by computer logic), to identify a physical head impact. In this example, the Teager-Kaiser energy operator measures instantaneous energy changes of signals composed of a single time-varying frequency. In some embodiments, extraneous sets of elements are removed so that the extracted position and element trajectories only include displacements relevant to a particular individual, surface, or object. An edge detection method, such as the Canny edge detection, can be used to identify the outline of an individual's head or helmet as illustrated in FIG. 5. A worker skilled in the art would appreciate that the Canny edge detection method is known in the art, and that other edge detection methods may be used without departing from the scope of the present disclosure. The weighted centroid (600 shown in FIG. 6) of all the pixels within the outline of an individual's head or helmet is calculated as illustrated in FIG. 6. The weighted centroid is used to determine the x, y coordinates of the center of the head in pixels over time, as illustrated in FIG. 7. In a preferred embodiment, scaling algorithms convert the smallest addressable element (pixel x, y coordinates) in a digital image to real-world object length and width measurements captured in the video. This is achieved by identifying objects in the frame of known size (e.g. boards, field markings, helmet size, goal post, ball) and calculating a pixel-to-real-world unit ratio. In another embodiment, elements of the field/rink/environment are localized in single or multiple images and used to transform two-dimensional pixel coordinates in the image to three-dimensional locations in real world units. Frame rate of the video capture can be assigned or converted to a predetermined frame per second rate. In an alternate embodiment, a physical head impact is detected using machine learning and/or statistical methods such as a CNN using a part or the entire field of view of a plurality of consecutive images that are captured in a video. The detection is accomplished by labeling a set of digital images (either in part or entire field of view) as containing or not containing a head impact. The models are trained by providing this set of images labeled with containing or not containing a head impact to one or more machine learning and/or statistical models.

With further reference to the third sub-step 203 in FIG. 2, the system extracts key characteristics of the head impact event. Key characteristics are often extracted using the set of elements of head and/or face, body parts (e.g. joints, segments) and objects and surfaces within an environment. The key characteristics can be identified by providing a set of digital images captured from a video as input to one or more machine learning models to yield a set of key elements of one or multiple individuals. Distinct individuals are recognized using discriminant features, including but not limited to jersey number, unit number, facial recognition. In a preferred embodiment, the key characteristics relate to the parameters that describe one or multiple physical head impact events. Indeed, when a physical head impact is detected, a set of event parameters are extracted from the images captured in a video. The event parameters are determined using the set of elements (head/helmet, body, objects, surfaces), captured from a video. A worker skilled in the art would appreciate that the event parameters would be determined using the position of a first set of elements in relation to the position of a second set of elements. The first set of elements includes the 2D or 3D position of head and body coordinates of a distinct individual relative to the position of coordinates of the second set of elements that includes, the 2D or 3D position of head and body coordinates of a different distinct individual or the environment. The environment includes surfaces, stationary and moving objects, projectiles, that are captured in a video. Object distance measurements are used to determine many of the head impact event parameters, within the preferred disclosure. This is accomplished using scaling algorithms and/or environment localization as previously described in the second sub-step 202. In an alternate embodiment, the key characteristics can relate to head and body resultant and/or axis kinematic components and/or waveforms resulting from one or multiple physical head impact events. In this alternate embodiment, the key characteristics would be determined using the position of a first set of elements in relation to the position of a second set of elements, where both the first and second set of elements include the 2D or 3D position of head and body coordinates of a distinct individual.

With further reference to the fourth sub-step 204 of FIG. 2, one or more machine learning models and/or statistical methods are used to predict a brain response. More specifically, the extracted key characteristics are used as input to a trained model to predict a brain response. The inputs (i.e. the key characteristics) comprise the event parameters of a head impact including, head impact velocity, location of impact on the head, mass of the bodies involved, direction and/or vector of the head impact, and the compliance/stiffness of the materials involved in the impact. Other inputs may also include head and body kinematic components or waveforms of one or multiple physical head impacts.

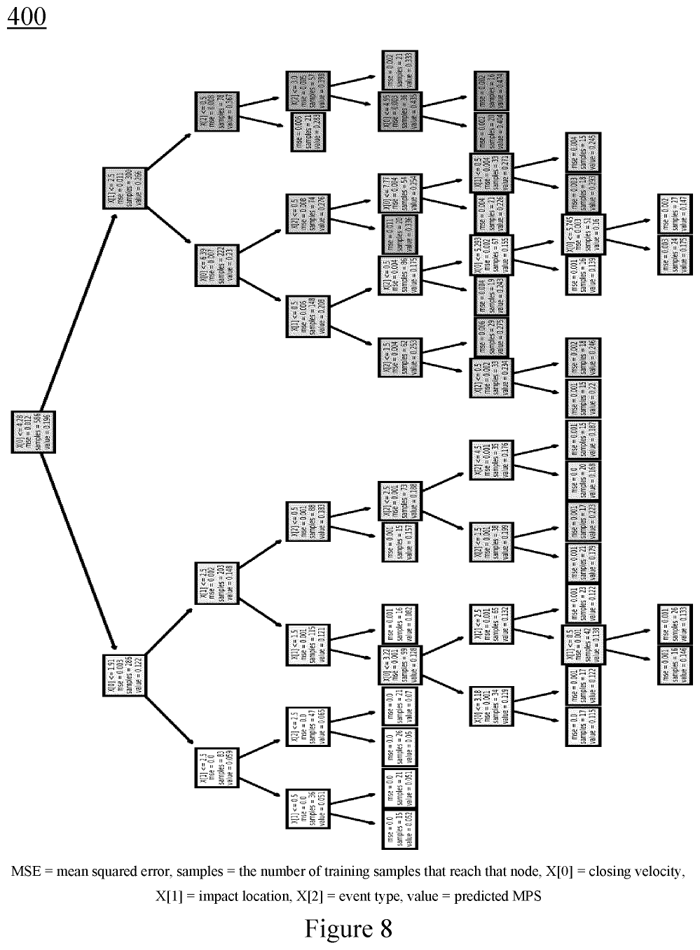

In an exemplary embodiment as shown in FIG. 8, a decision tree 400 is used to predict maximum principal strain as the brain response. The decision tree 400 is trained for this regression problem and uses mean square error to measure quality of the split, with a strategy of using the best split. The minimum samples per split was set to 15 samples and there was no maximum depth, minimum number of samples required to be a leaf node, maximum number of leaf nodes, minimum weight fraction of the sum total of weights required to be a leaf node, or maximum number of features to consider when looking for best split set before training. In addition, there was no early stopping in tree growth nor pruning. To train and test the model, an 80:20 split test-train ratio can be used and to assess generalizability, 10-fold cross-validation is performed. Predictions can be treated as either a regression problem (predicting the brain response value) or a classification problem (predicting the class of brain response (e.g. very low, low, medium, high, very high). To ensure all training data with different units are using the same scale, the data from the extracted set of characteristics may be transformed, for example, using a one-hot encoder into bit arrays, or using a StandardScaler.

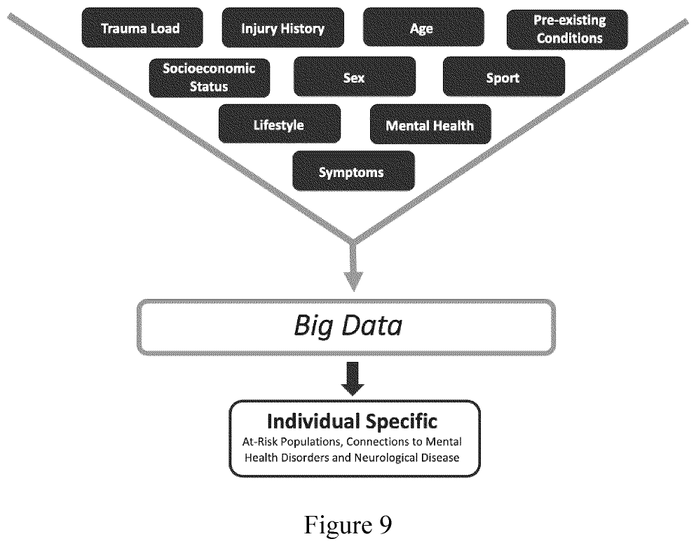

With further reference to the third step 103 of FIG. 1, one or more machine learning models and/or statistical methods are used to generate brain trauma assessments using brain response measurements. In a preferred embodiment, the brain trauma assessments are performed using the predicted brain response caused from one or multiple head impact events and relate to the magnitude, level, severity and/or risk to injury. Severity and risk associated with one or multiple head impacts associate with neurological injury and brain damage, which may include concussion, sub-concussion, cognitive impairments, mental health disorders and brain disease. Brain trauma assessments include high-risk identification, brain trauma profile, and evaluation scores, produced using one or multiple head impact events. High-risk identification is generated based on the predicted brain response of a single head impact event or an accumulation from multiple head impact events. In a preferred embodiment, a high-risk is an indication of the brain response value exceeding a predetermined threshold. In the present embodiment, the predicted brain response is also used to produce brain trauma profiles using additional characteristics common to environments where head impacts are recurrent. Examples of such characteristics include the number of head impacts, the magnitude of head impacts, or the time interval between the head impacts. In other embodiments, the brain trauma profile may incorporate the set of event parameters, for example, the event type or frequency of event type, the impact location or frequency of impact location. The brain trauma profile is generated over a specified duration of time, such as, a game, match, day, week, season, year. In the present invention, machine learning is used to generate evaluation scores for individuals or groups that share a set of features. The evaluation scores are generated using a specified set of features, including but not limited to an activity, sport, gender, sex, age, ability, level or division of competition. In the preferred embodiment of the disclosure, the evaluation scores are generated from multiple features of an individual. In the example illustrated in FIG. 9, a set of features are used to generate large data sets to provide individualized evaluation scores. In the disclosure, the evaluation scores are of one or multiple of the set of features.

With further reference to the fourth step 104 of FIG. 1, the system provides outputs of the generated brain trauma assessments as indications of brain trauma exposure. One skilled in the art would appreciate that exposure to brain trauma can be measured in a variety of ways and therefore is not limited to the examples of brain trauma assessments provided in this embodiment. Single and accumulative brain trauma assessments are provided as measures of exposure for one or more individuals and evaluated as an individual or team or group of individuals. In some embodiments, the output includes a comparison between preceding brain trauma exposure of distinct individuals or multiple individuals. The system conveys to the user, who is that of an individual, group, organization or system, brain trauma assessments based on one or multiple head impact events. The outputs are used by players, parents of participants, sports teams, medical staff, organizations or systems. Application of brain trauma assessments can include tracking and monitoring brain trauma, managing brain trauma loads for one or multiple individuals, evaluating coaching techniques and play strategy, and modification of rules and policies that govern safe sport regulation. The brain trauma assessments of one or multiple head impacts may be used by medical professionals and clinicians to aid in diagnosis and/or prognosis of head injury, treatment plans and return to sport, activity or work protocols. Brain trauma assessments are provided to the user in real-time and/or post processing.

With further reference to FIG. 3 and according to an embodiment of the present disclosure, a system 300 is provided, the system 300 for determining and evaluating brain trauma exposure. In a preferred embodiment, the system 300 is comprised of at least one motion capture source to record athletes playing a game such as ice hockey. In this example, four cameras 301, 302, 303, 304 are used. The video cameras 301, 302, 303, 304 are connected to a non-transitory computer readable medium such as a computer or mobile device 320, where video footage is uploaded into a user portal on the frontend. A worker skilled in the art would appreciate that in another embodiment, the cameras 301, 302, 303, 304 may be wirelessly connected to the cloud or to another type of remote processing platform that can process a series of instructions based on the video taken from the cameras 301, 302, 303, 304. Video footage is then provided as input data to one or more server 330 in the backend, to process images of the ice hockey game taken from the cameras 301, 302, 303, 304. Once the video has been received by the server 330, the server 330 is configured to extract key characteristics from the video, predict a brain response based on those key characteristics and provide, back to the user portal on the frontend computer 320, a brain trauma assessment output data based on the brain response. In a preferred embodiment, the brain trauma assessment output is sent wirelessly to a remote device such as a cellular phone (not shown) to a user, or over a network to another computer (not shown) or source capable of displaying the data.

With reference to FIGS. 1, 2 and 3 and according to an embodiment of the present disclosure, a practical example of steps 101, 102, 103, and 104 of method 100 are described in greater detail. In the first step, 101, ground truth datasets of head impacts occurring in ice hockey are used to train a machine learning model. The key characteristics of head impact event parameters including impact velocities, locations and event types are used as input with a known brain response output, specifically, the amount of brain tissue displacement as a measure of maximum principal strain. Although the displacement is the brain response measurement in this example, one skilled in the art can appreciate other types of responses which may not be displacement are able to be measured. A minimum of 1000 (700 train, 150 validate, 150 test) head impacts are used to train the prediction model. In the first sub-step 201 of step 102, a hockey game is taking place and being captured on video, in real-time. A system 300 is receiving video input 320 from four cameras 301, 302, 303, 304 set up around an ice rink 310. In the second sub-step 202, the system 330 identifies and tracks players 305 and environment within the ice rink. In this exemplary embodiment, the environment is the hockey net 306, and boards 307; however, other objects within the environment can be identified. This identification is accomplished using a set of elements, which are the 2D coordinates of the head and body, objects and surfaces (as shown in FIG. 4). In the second sub-step 202, the system 330 then detects a head impact using the set of elements. In this example, the detection occurs by calculating the change of the velocity of the head as it moves from one frame compared to the next frame(s). In the third sub-step 203, the system 330 calculates that the player 305 impacted the side of their head against their opponent's shoulder, at a velocity of 2.3 m/s. The system 330 uses the position of the 2D coordinates to extract these parameters of the head impact event. In the fourth sub-step 204, the magnitude of the brain response is calculated by inputting the event parameters of 1-side of the head, 2-head hit against shoulder, 3-velocity of 2.3 m/s, into a machine learning model. In this example, the model is a decision tree trained to predict the maximum principal strain the brain experiences from the head impact (as shown in FIG. 8). The model predicts a 30% strain, which is classification of ‘high’ severity. Once the brain response has been predicted, the system 330 can generate a brain trauma assessment 325 in step 103. In this example, the 30% predicted brain response is above a predetermined threshold of 28%, identifying a high-risk head impact has been received. In the final step 104, the brain trauma assessment is output 320 to the end user, in this example, the sideline staff 308, is sent an alert of a high-risk identification.

Although many examples of the present disclosure described are with reference to sport and physical activity, the collection of brain trauma exposure can be used in variety of applications, such as in the military and workplace. Further, the processes outlined for evaluating brain trauma exposure can be used within a variety of fields contributing to commercial, scientific, academic and medical sectors. For example, processes in accordance with this method, might be ready modified to include additional personal characteristics of the individual that could affect their risk. The system has the ability to accept additional features of the individual, as illustrated in FIG. 9, so the outputs become more personalized. With the inclusion of added personal features (e.g. head injury history, pre-existing condition or disorder (e.g. ADHD, depression), socioeconomic status, lifestyle), predication algorithms can be refined. As the system collects and analyzes thousands of head impacts, one versed in the art can appreciate that the system will be become smarter and a more individualized report of the risks associated with brain trauma exposure will be produced. The collection of brain trauma exposure can also be used in conjunction with mental health, cognitive, brain injury and disease measures to research relationships between brain trauma exposure and clinical outcomes. The predicted brain response may also be broadened to include measurements of any biological, physical, mechanical and/or chemical components that associate with neurological damage, brain injury and/or brain disease. Finally, head impacts and brain trauma exposure tracking and monitoring can be used for purposes other than described in the present disclosure, such as broadcasting, gambling, playing and coaching style, contextual scenario, officiating and rule enforcement.

Many modifications of the embodiments described herein as well as other embodiments may be evident to a person skilled in the art having the benefit of the teachings presented in the foregoing description and associated drawings. It is understood that these modifications and additional embodiments are captured within the scope of the contemplated disclosure, which is not to be limited to the specific embodiment disclosed.

1. A method for evaluating brain trauma exposure, the steps comprising:
extracting key characteristics from at least one motion capture source;
predicting a brain response based on the key characteristics; and,
providing a brain trauma assessment output based on the brain response,
wherein at least one of statistical methods and machine learning models are utilized to predict the brain response and provide the brain trauma assessment.

2. The method of claim 1, wherein the step of extracting key characteristics further comprises:
identifying individuals, the individuals performing a set of movements; and,
detecting a physical head impact of at least one of the individuals; and,
measuring at least one of: at least one head impact event parameter and head kinematic components, based on the detected physical head impact.

3. The method of claim 2, wherein the step of detecting the physical head impact further comprises:
a. identifying a first set of coordinates of key elements from a first frame of the at least one motion capture source;
b. identifying a second set of coordinates of key elements from a second subsequent frame of the at least one motion capture source;
c. computing a difference in coordinates of key elements; and,
d. repeating steps a. to c. until a physical head impact is detected.

4. The method of claim 3 further comprising using an energy operator to compute the difference in coordinates of the key elements.

5. The method of claim 2, wherein the step of detecting the physical head impact further comprises:
providing a set of digital images captured from the at least one motion capture source to a first machine learning model to train the first machine learning model to detect the physical head impact,
wherein the set of digital images are pre-identified as one of: having a head impact and not having a head impact.

6. The method of claim 2, wherein the step of measuring at least one of: at least one head impact event parameter and head kinematic components further comprises:
a. identifying a first set of coordinates of key elements from a first frame of the at least one motion capture source;
b. identifying a second set of coordinates of key elements from a second subsequent frame of the at least one motion capture source;
c. computing a difference in coordinates of key elements; and,
d. repeating steps a. to c. until one of: the at least one head impact event parameter and the head kinematic components is measured.

7. The method of claim 1, wherein the brain trauma assessment is one of: a customizable brain trauma evaluation score, a brain trauma profile, and a high risk brain identification, provided to a user.

8. A system for evaluating brain trauma exposure, the system comprising:
at least one motion capture source; and,
a non-transitory computer readable medium connected to the at least one motion capture source, the computer readable medium configured to:
extract key characteristics from the at least one motion capture source;
predict a brain response based on the key characteristics; and,
provide a brain trauma assessment output based on the brain response
wherein at least one of statistical methods and machine learning models are utilized to predict the brain response and provide the brain trauma assessment,
and wherein the brain trauma exposure is provided to a user.

9. The method of claim 8, wherein the step of extracting key characteristics further comprises:
identifying individuals, the individuals performing a set of movements; and,
detecting a physical head impact of at least one of the individuals; and,
measuring at least one of: at least one head impact event parameter and head kinematic components, based on the detected physical head impact.

10. The method of claim 9, wherein the step of detecting the physical head impact further comprises:
a. identifying a first set of coordinates of key elements from a first frame of the at least one motion capture source;
b. identifying a second set of coordinates of key elements from a second subsequent frame of the at least one motion capture source;
c. computing a difference in coordinates of key elements; and,
d. repeating steps a. to c. until a physical head impact is detected.

11. The method of claim 10 further comprising using an energy operator to compute the difference in coordinates of the key elements.

12. The method of claim 9, wherein the step of detecting the physical head impact further comprises:
providing a set of digital images captured from the at least one motion capture source to a first machine learning model to train the first machine learning model to detect the physical head impact,
wherein the set of digital images are pre-identified as one of: having a head impact and not having a head impact.

13. The method of claim 9, wherein the step of measuring at least one of: at least one head impact event parameter and head kinematic components further comprises:
a. identifying a first set of coordinates of key elements from a first frame of the at least one motion capture source;
b. identifying a second set of coordinates of key elements from a second subsequent frame of the at least one motion capture source;
c. computing a difference in coordinates of key elements; and,
d. repeating steps a. to c. until one of: the at least one head impact event parameter and the head kinematic components is measured.

14. The method of claim 8, wherein the brain trauma assessment is one of: a customizable brain trauma evaluation score, a brain trauma profile, and a high risk brain identification, provided to a user.
